(19) United States
(12) Patent Application Publication
(10) Pub. No.: US 2024/0210735 A1
(43) Pub. Date: Jun. 27, 2024

(54) OPTHALMIC LENS FOR MYOPIA CONTROL

(71) Applicant: Johnson & Johnson Vision Care, Inc., Jacksonville, FL (US)

(72) Inventors: Minghan Chen, Jacksonville, FL (US); Noel A Brennan, Jacksonville, NJ (US); Xu Cheng, Jacksonville, FL (US); Jaclyn Hernandez, Jacksonville, FL (US); Emily Hester-Hotte, Jacksonville, FL (US)

(21) Appl. No.: 18/537,862

(22) Filed: Dec. 13, 2023

Related U.S. Application Data
(60) Provisional application No. 63/434,819, filed on Dec. 22, 2022.
(60) Provisional application No. 63/548,432, filed on Nov. 14, 2023.

(51) Int. Cl. G02C 7/06

(52) U.S. Cl. CPC G02C 7/06; G02C 2202/24

(57) ABSTRACT

An ophthalmic lens for slowing or preventing the progression of myopia having a lens center, a shape defined by a lens outer peripheral edge, and an optic zone surrounding lens center and having an outer periphery. The optic zone has a central optic zone surrounding the lens center and defined by a central optic zone radius, and a peripheral optic zone surrounding the central optic zone and extending to the optic zone outer periphery. The lens includes a plurality of myopic defocus zones each positioned at a predetermined radial distance from the lens center, and each introducing a predetermined ADD power that increases with increased radial distance from lens center up to a radius of at least 4 mm. At least a first myopic defocus zone is positioned within the central optic zone, and at least first and second myopic defocus zones are positioned in the peripheral optic zone.

### CROSS-REFERENCE TO RELATED APPLICATIONS

The present application claims priority to U.S. Provisional Application No. 63/434,819, filed on Dec. 22, 2022, and U.S. Provisional Application No. 63/548,432, filed on Nov. 14, 2023.

### FIELD OF THE INVENTION

The present application relates to an ophthalmic lens designed to arrest or reduce the progression of myopia in a patient. More specifically, the present application is directed to lens designs and methods for lens design that induce a predetermined peripheral blur orientation or that otherwise increase average peripheral blur anisotropy values at the retinal plane.

### BACKGROUND

Common conditions which lead to reduced visual acuity include myopia and hyperopia for which corrective lenses in the form of spectacles, rigid or soft contact lenses are prescribed. The conditions are generally described as the imbalance between the length of the eye and the focus of the optical elements of the eye. Myopic eyes focus light from distant objects in front of the retinal plane, and unaccommodated hyperopic eyes focus light from distant objects behind the retinal plane. Myopia typically develops because the axial length of the eye grows to be longer than the focal length of the optical components of the eye, that is, the eye grows too long. Hyperopia typically develops because the axial length of the eye is too short compared with the focal length of the optical components of the eye.

Myopia has a high prevalence rate in many regions of the world. Of greatest concern with this condition is its possible progression to high myopia, for example, greater than five (5) or six (6) diopters, which dramatically affects one's ability to function without optical aids. High myopia is also associated with an increased risk of retinal disease, cataract, glaucoma, and myopic macular degeneration (MMD; also known as myopic retinopathy) and may become a leading cause of permanent blindness worldwide. MMD has been related to refractive error (RE) to a degree rendering no clear distinction between pathological and physiological myopia and such that there is no “safe” level of myopia.

Corrective lenses are used to alter the gross focus of the eye to render a clearer image at the retinal plane, by shifting the focus from in front of the plane to correct myopia, or from behind the plane to correct hyperopia, respectively. However, the corrective approach to the conditions does not address the cause of the condition, but rather is merely prosthetic or intended to address symptoms.

Most eyes do not have simple myopia or hyperopia, but also have myopic astigmatism or hyperopic astigmatism. Astigmatic errors of focus cause the image of a point source of light to form as two mutually perpendicular lines at different focal distances. In the following discussion, the terms myopia and hyperopia are used to include simple myopia and myopic astigmatism and hyperopia and hyperopic astigmatism respectively.

Emmetropia describes the state of clear vision where an object at infinity is in relatively sharp focus without the need for optical correction and with the crystalline lens relaxed. In normal or emmetropic adult eyes, light from both distant and close objects passing through the central or paraxial region of the aperture or pupil is focused by the crystalline lens inside the eye close to the retinal plane where the inverted image is sensed. It is observed, however, that most normal eyes exhibit positive longitudinal spherical aberration, meaning that rays passing through the aperture or pupil at its periphery are focused in front of the retinal plane when the eye is focused to infinity. As used herein the measure D is the dioptric power, defined as the reciprocal of the focal distance of a lens or optical system, in meters.

The spherical aberration of the normal eye is not constant. For example, accommodation (the change in optical power of the eye derived primarily through changes to the crystalline lens) causes the spherical aberration to change from positive to negative.

As noted, myopia typically occurs due to excessive axial growth or elongation of the eye. It is now generally accepted that axial eye growth can be influenced by the focus and quality of the retinal image, and that altering the retinal image can lead to consistent and predictable changes in eye growth.

Known approaches that attempt to eliminate or reduce axial elongation of the eye, particularly in children, include the application of ophthalmic lenses that intentionally introduce myopic defocus in the field of vision. The myopic defocus introduces a ‘stop’ stimulus to the eye that results in limitation of eye growth. This is initially observed as a thickening of the choroid. Eye growth changes in response to retinal image defocus have been demonstrated in animal studies to be largely mediated through local retinal mechanisms, because eye length changes still occur when the optic nerve is damaged, and because imposing defocus on local retinal regions has been shown to result in altered eye growth localized to that specific retinal region.

Ophthalmic lenses with concentric annular designs have been shown to slow myopia progression. These include the Acuvue® Bifocal lens by Johnson & Johnson Vision Care, Inc. and the MiSight® contact lenses by CooperVision, Inc. These lenses have certain annular zones which contain optics that correct for myopia, while others introduce myopic defocus. Light from distant objects along the optical axis that pass through a given annulus essentially comes to a point focus on the optical axis; on the retina and in front of the retina for the myopic correction annuli and myopic defocus annuli, respectively.

U.S. Pat. No. 10,901,237, which is incorporated herein by reference in its entirety, describes various other lens designs for myopia control, with particular application in soft contact lenses. These lenses also have a concentric annular design where certain annular sections include optics that focus on the retina. For patients that require correction for myopia, these annular sections may include optics that redirect the focal point onto the retina. For patients that do not require myopia correction, these annular sections may provide no optical correction. Myopic defocus annular sections that are not centrally located contain optics that cause light passing therethrough to focus in front of the retina, but rather than a point focus on the optical axis, the light forms a non-coaxial ring focus. In some disclosed embodiments, a central portion of the lens contains an add power that induces myopic defocus but along the optical axis.

Lens designs such as these tend to emphasize on-axis optics, that is from rays that originate from objects along the optical axis, and more particularly create foci with respect to the central region. Further, these lens designs are focused on a central-vision-based design principle and incorporate optical elements within the central vision area to generate myopic defocus. In addition to central vision, it has been shown that peripheral refractive errors can have a substantial impact on central refractive development, and myopic defocus in the near periphery can slow axial growth. See Smith, Vision Reg., September 2009; 49(19): 2386-2392).

Additional aberrations occur in peripheral vision that may further contribute to myopia progression. In astigmatic eyes, refraction of the eye is not spherical or rotationally uniform, but rather because of the oblong or “football” shape of the eye, refractive power along one meridian is different than that along the perpendicular meridian, such that focal lines along the two meridians are at different distances. Regular astigmatism is typically categorized as “with the rule”, “against the rule” or “oblique”. For individuals with “with the rule” astigmatism (most common), the vertical meridian has the strongest power or steepest curvature as compared to the horizontal meridian. Oblique astigmatism occurs when the strongest power or steepest curvature does not fall on the vertical or horizontal meridians.

As well as the potential for astigmatism in central vision, it is well known that peripheral astigmatism is created by rays striking the refracting surfaces of the cornea and crystalline lens at oblique incidence. This astigmatism and other wavefront aberrations cause optical blur in the peripheral retina that is highly anisotropic. Blur anisotropy is defined herein as the logarithm (base 10) of the ratio of the area under the modulation transfer function (MTF) along the vertical meridian (superior-inferior meridian) divided by the area under the MTF along horizational (temple-nasal) meridian, that is, blur anisotropy=log[(area)_{vertical}/(area)_{horizational}]. As a result, blur anisotropy is negative when the image (or point spread function) has a predominantly vertical orientation blur and is positive when the image has a predominantly horizontal orientation blur. The human neural system is more sensitive to horizontally oriented visual stimuli than vertically oriented stimuli. Further, the orientation of the blur pattern created by astigmatism varies from horizontal to vertical moving around the periphery of the retina, a principle known as the meridional effect.

An exemplary illustration of peripheral blur anisotropy is shown in FIG. 2, where blur at the retinal plane has a non-symmetric pattern such that the length of the blur pattern in one direction (i.e., along the y-axis) is longer than in the perpendicular direction. In FIG. 2, optical resolution will be finer along the x axis than along the y axis. As noted, the orientation of the peripheral blur will change around the periphery of the retina.

The peripheral visual field may also be leveraged to introduce myopia control effects. In a recent publication entitled “Eccentricity-Dependent Effects of Simultaneous Competing Defocus on Emmetropization in Infant Rhesus Monkeys” (Vision Research 177 (2020) 32-40), the authors showed that myopic defocus out to about 20° from the fovea can substantially impact central refractive development in primates. In another publication, it was hypothesized that the neural system's orientation sensitivity coincides with habitual blur orientation, and that blur orientation may be a trigger for eye growth. Zhelenznyak et al. (2016), Optical and Neural Ansiotropy in Peripheral Vision, Journal of Vision, 16(5):1, 1-11.

More recently, Ji et al. leveraged modeling techniques to evaluate certain bifocal and multifocal contact lens adapted to incorporate add power for inducing myopic defocus, in comparison to monofocal lenses, to assess the impact of the lenses on peripheral vision at various eccentricities. Ji et al., Through-focus Optical Characteristics of Monofocal and Bifocal Soft Contact Lenses Across the Peripheral Visual Field, Ophthalmic & Physiological Optics, 38 (2018) 326-336. The results of the study indicated that the former two lenses, when compared to the monovision lens, demonstrated a decrease in anisotropy of peripheral optical blur and also an increased depth of focus. From this, the authors hypothesized that the mechanism underlying myopia control with the bifocal and multifocal lenses was the decrease in anisotropy of peripheral optical blur coupled with the increase in depth of focus.

U.S. Patent Publication No. 2022/0252901 to Yoon (“Yoon”) describes optical lenses for myopia control that intentionally introduce peripheral aberrations for the purposes of manipulating peripheral blur to make it more radially symmetric. In other words, based off the earlier hypothesis in the Ji paper that decreasing anisotropy of peripheral optical blur is desired, the application describes the objective of intentionally minimizing anisotropy of the peripheral region to the point where it is radially symmetric, for example, as shown in FIG. 1. In other words, the lenses described in Yoon prefer to have the circle of least confusion at the patient's retina. It means one focus line is behind retina and one focus line is in front of retina. To the contrary, the lenses described herein prefer that both the tangential and sagittal focus lines are in front of the retina or have an adjusted blur anisotropy value toward the positive direction.

The inventors herein have discovered that, contrary to the teachings of Yoon and Ji, lenses designed with high blur anisotropy of the opposite orientation to that found with peripheral hyperopia exhibit better myopia control treatment efficacy. Leveraging these findings, lens designs that incorporate peripheral optics that increase the average peripheral blur anisotropy values (toward positive direction) will also improve efficacy. The present disclosure is directed to such lens designs and methods for designing such lenses.

### SUMMARY OF THE INVENTION

An ophthalmic lens is provided for at least one of slowing or preventing the progression of myopia. The lens includes a lens center and a shape defined by a lens outer peripheral edge, an optic zone surrounding the lens center and having an optic zone outer periphery, where the optic zone further includes a central optic zone surrounding the lens center and defined by a central optic zone radius, and a peripheral optic zone surrounding the central optic zone and extending to the optic zone outer periphery. The lens further includes a plurality of myopic defocus zones each positioned at a predetermined radial distance from the lens center, and each introducing a predetermined ADD power, where the predetermined ADD power for the plurality of myopic defocus zones increases with increased radial distance from lens center up to a radius of at least 4 mm, and wherein at least a first myopic defocus zone is positioned within the central optic zone, and at least first and second myopic defocus zones are positioned in the peripheral optic zone.

In alternate embodiments, the central optic zone radius may be between 4 and 5 mm, and/or the at least one myopic defocus zone positioned within the central optic zone may be located at least at a radial distance of greater than 1.2 mm from lens center. The at least one myopic defocus zone in the central optic zone further may have an ADD power of at least 1.5D.

In another embodiment, the first and second myopic defocus zones positioned in the peripheral optic zone may be located at least at radial distances of about 2.25 mm and 3.0 mm respectively from lens center, and may further have ADD powers of about 3.0 and 4.0 respectively.

In yet another embodiment, the lens further includes a fourth and a fifth myopic defocus zone positioned in the peripheral optic zone, and the fourth myopic defocus zone may be positioned at a radial distance of at least 3.75 mm from lens center, and the fifth myopic defocus zone may be positioned at a radial distance of at least 4.5 mm from lens center. The fourth myopic defocus zone may have an ADD power of at least about 5D, and the fifth myopic defocus zone may ahve an ADD power of at least 6D.

In yet another embodiment, the myopic defocus zones in the central and peripheral optic zones may each cause light passing therethrough to generate non-coaxial myopic ring defocus.

In an alternate embodiment, the least one myopic defocus zone positioned within the central optic zone is located at least at radial distance of greater than 1.2 mm from lens center, and may further have an ADD power of at least 3D.

In this embodiment, the at least first and second myopic defocus zones positioned in the peripheral optic zone may be located at radial distances of at least about 2.56 mm and 3.22 mm respectively from lens center, and may further have ADD powers of about 8D and 9.8D respectively.

This lens may further include a fourth and a fifth myopic defocus zone positioned in the peripheral optic zone, which may be positioned at radial distances of at least about 3.87 mm from lens center and about 4.53 from lens center, and which may both further have an ADD power of at least 12D.

The lens may also further include a second myopic defocus zone positioned at least partially within the central optic zone, which may be positioned at a radial distance of at least about 1.91 mm from lens center and may have an ADD power of at least about 5.9D.

The myopic defocus zones in the central and peripheral optic zones may each cause light passing therethrough to generate non-coaxial myopic ring defocus.

Also provided is an ophthalmic lens for at least one of slowing or preventing the progression of myopia which includes a lens center, and a shape defined by a lens outer peripheral edge, an optic zone surrounding the lens center and having an optic zone outer periphery, where the optic zone further includes a central optic zone surrounding the lens center and defined by a central optic zone radius, and a peripheral optic zone surrounding the central optic zone and extending to the optic zone outer periphery. The lens further includes a plurality of myopic defocus zones each positioned at a predetermined radial distance from the lens center, and each introducing a predetermined ADD power, wherein each of the at least first and second myopic defocus zones positioned in the peripheral optic zone have a larger ADD power than the at least first myopic defocus zone positioned within the central optic zone.

In one embodiment, the central optic zone radius int his lens is between 4 and 5 mm, and the at least one myopic defocus zone positioned within the central optic zone may be located at least at radial distance of greater than 1.2 mm from lens center, and may have an ADD power of at least 1.5D.

In another embodiment, the first and second myopic defocus zones positioned in the peripheral optic zone may be located at least at radial distances of about 2.25 mm and 3.0 mm respectively from lens center, and may further have ADD powers of about 3.0D and 4.0D respectively.

In yet another embodiment, the lens further includes a fourth and a fifth myopic defocus zone positioned in the peripheral optic zone, which may be positioned at a radial distance of at least 3.75 mm and 4.5 mm from lens center respectively, and may have ADD powers of at least about 5D and at least 6D respectively.

The myopic defocus zones in the central and peripheral optic zones may each cause light passing therethrough to generate non-coaxial myopic ring defocus.

In yet another embodiment, the at least one myopic defocus zone positioned within the central optic zone may be located at least at radial distance of greater than 1.2 mm from lens center, and may further have an ADD power of at least 3D. The at least first and second myopic defocus zones positioned in the peripheral optic zone may be located at radial distances of at least about 2.56 mm and 3.22 mm respectively from lens center, and may further have ADD powers of about 8D and 9.8D respectively.

In yet another embodiment, the lens further includes a fourth and a fifth myopic defocus zone positioned in the peripheral optic zone, and the fourth myopic defocus zone may be positioned at a radial distance of at least about 3.87 mm from lens center, and the fifth myopic defocus zone may be positioned at a radial distance of at least about 4.53 from lens center, and the fourth and fifth myopic defocus zones may further have an ADD power of at least 12D.

In yet another embodiment, the lens further includes a second myopic defocus zone positioned at least partially within the central optic zone, which may be positioned at a radial distance of at least about 1.91 mm from lens center and may further have an ADD power of at least about 5.9D.

The myopic defocus zones in the central and peripheral optic zones may each cause light passing therethrough to generate non-coaxial myopic ring defocus.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 illustrates an anisotropic peripheral blur pattern; where the illustrated pattern is generated by ray tracing through a model eye where each cross represents the intersection of a ray with the retina, and where rays originating at a point source are evenly distributed throughout the pupil.

FIG. 2 illustrates an anisotropic peripheral blur pattern where the pattern is generated by ray tracing through a model eye where each cross represents the intersection of a ray with the retina, and where rays originating at a point source are evenly distributed throughout the pupil.

FIG. 3 illustrates an eye model for simulating center and peripheral vision;

FIGS. 4(a)-(d) illustrate blur anisotropy values for various lenses;

FIG. 10 illustrates an exemplary lens to which the present inventions can be applied;

FIG. 11 illustrates power profiles for lenses according to the present disclosure that incorporate peripheral optic features;

FIGS. 12a and 12b illustrate blur anisotropy values for the embodiments of FIG. 11 under 4 mm pupil size conditions;

FIGS. 13a and 13b illustrate blur anisotropy values for the embodiments of FIG. 11 under 5 mm pupil size conditions; and

FIGS. 14a and 14b illustrate the blur anisotropy values for the lenses of FIGS. 4b and 4d under 5 mm pupil size conditions.

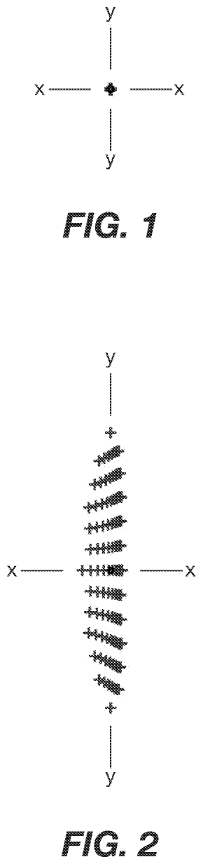

### DETAILED DESCRIPTION OF THE INVENTION

The present disclosure relates to ophthalmic lens designs and methods for ophthalmic lens design that induce directional peripheral blur to increase myopia control efficacy. The present disclosure also relates to ophthalmic lens designs and methods for lens designs that introduce optical elements or features into the peripheral optic zone that increase average peripheral blur anisotropy values to thereby increase myopia control efficacy or otherwise provide a better balance of efficacy and visual acuity. Ophthalmic lenses for which the disclosure applies include, but are not limited to, contact lenses to be worn on the eye of a person or wearer, spectacle lenses, and implantable ophthalmic lenses such as intraocular lenses and phakic lenses.

### Variable Toric Designs

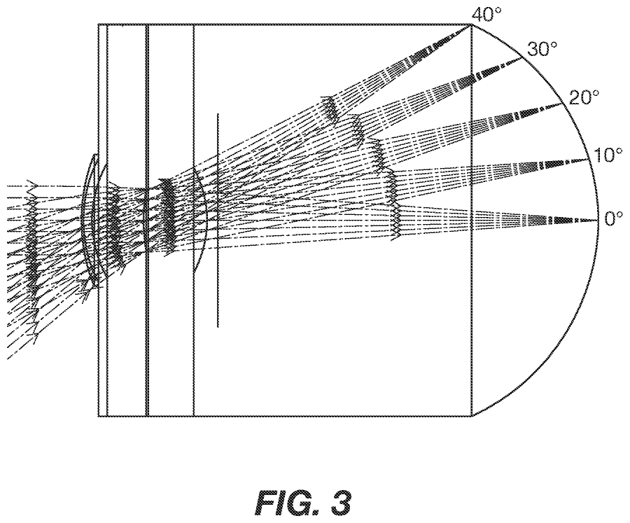

As noted above, typical human vision provides in-focus images in the central vision area and increasingly anisotropic blurred images toward the periphery of the eye. For individuals in need of correction (myopia or hyperopia), ophthalmic lenses focus on correction of central vision, which typically is within about 10 degrees of the line of sight. FIG. 3 illustrates an eye model showing field of view (FOV) ranging from 0 degrees to 40 degrees. This eye model used herein to assess peripheral blur is similar to the model described in detail by Navarro et al. in the publication J. Opt. Soc. Am. A 16, 1881-1891 (1999), which is incorporated herein by reference. The model has been modified only to include slight variations in the corneal and crystalline lens surfaces of the model based on using actual biometric data. The eye model was constructed to represent the averaged ocular optical and mechanical properties including wavefront aberrations, anterior corneal curvature, etc. of the general population. The eye model was also used for visual disturbance (scattering) simulation as published described by Chen in the publication Evaluating the Effects of Scattering on Retinal Image Quality, Proc. SPIE 11941, Ophthalmic Technologies XXXII, 119410B (4 Mar. 2022).

It is known to those skilled in the art that in the typical eye, blur increases at larger peripheral FOV angles. It is further known from the meridional effect, that the orientation of the blur pattern varies around the periphery. Furthermore, along the horizontal meridian, there is better performance for higher FOV angles for a viewed horizontal grating than for a vertical grating when compared to central vision which sees substantially similar images.

As also noted above, Ji hypothesized that eliminating or minimizing peripheral blur orientation (creating an isotropic blur pattern) will improve myopia control efficacy in a lens. Contrary to this teaching, the inventors herein have discovered that it is desirable and more efficacious to induce an anisotropic blur pattern formed by myopic astigmatism.

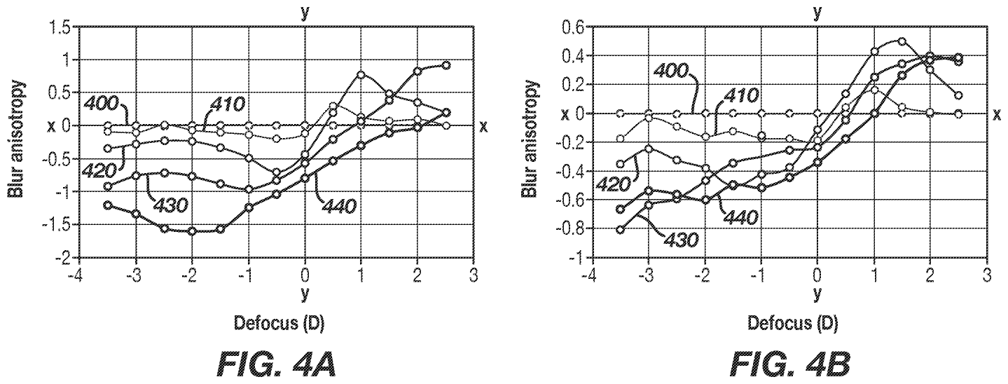

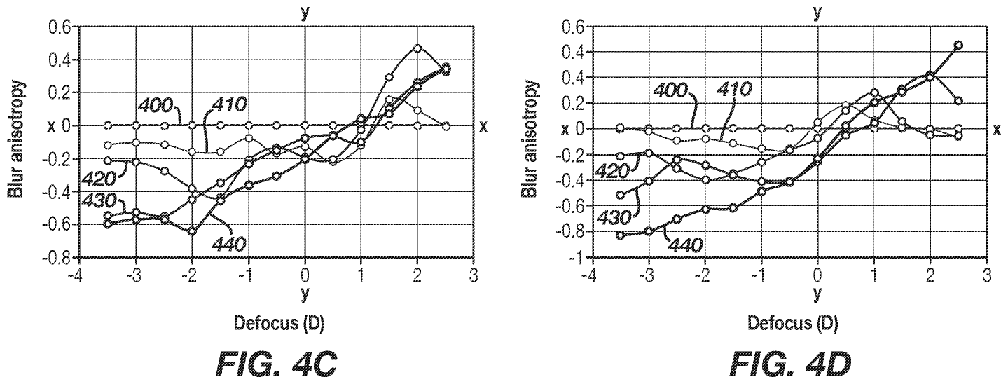

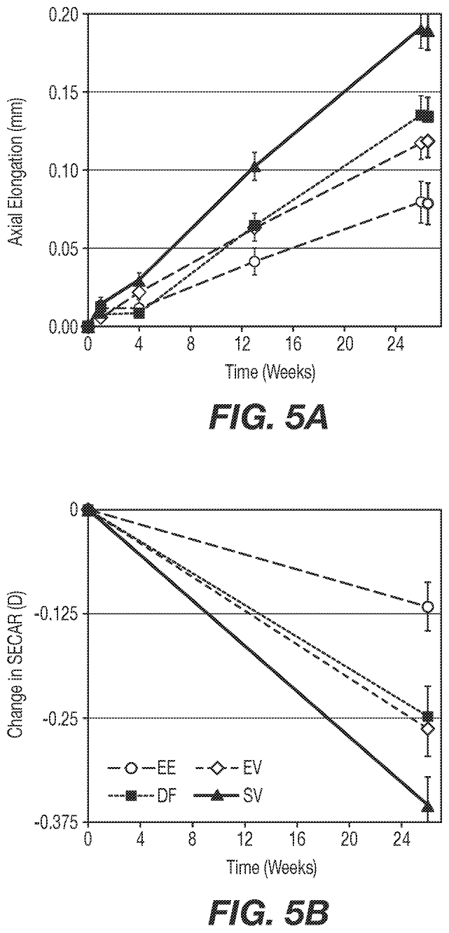

To quantitatively evaluate the peripheral image blur orientation, several myopia control lenses, including the Acuvue® Abiliti™ lens from Johnson & Johnson Vision Care, Inc., were evaluated leveraging the models described above under 4 mm pupil conditions. FIGS. 4a-4b illustrate the blur anisotropy of these lenses as evaluated, where the single vision lens (“Lens 1”) is shown in FIG. 4a, the Acuvue® Abiliti™ lens (“Lens 4”) is shown in FIG. 4d, a first test lens similar to the Acuvue® Abiliti™ lens but without a high add power center treatment zone is shown in FIG. 4b (“Lens 2”), and a second, dual focus test lens with dual co-axial focal points is shown in FIG. 4c (“Lens 3”). Blur anisotropy is negative when the image has a vertical orientation and is positive when the image has a horizontal orientation. Within each figure, blur anisotropy at 0, 10, 20, 30 and 40 degree FOV are shown by reference numerals 400, 410, 420, 430 and 440, respectively. For each graph, the vertical line at x=0 represents the retinal plane, or zero defocus, and the horizontal axis indicates defocus either in front of the retina (positive) or behind (negative). As shown in these figures, for all lenses the blur anisotropy is negative at the retinal plane for all degrees FOV other than zero, except for at 10 degrees FOV for the lens of FIG. 4d.

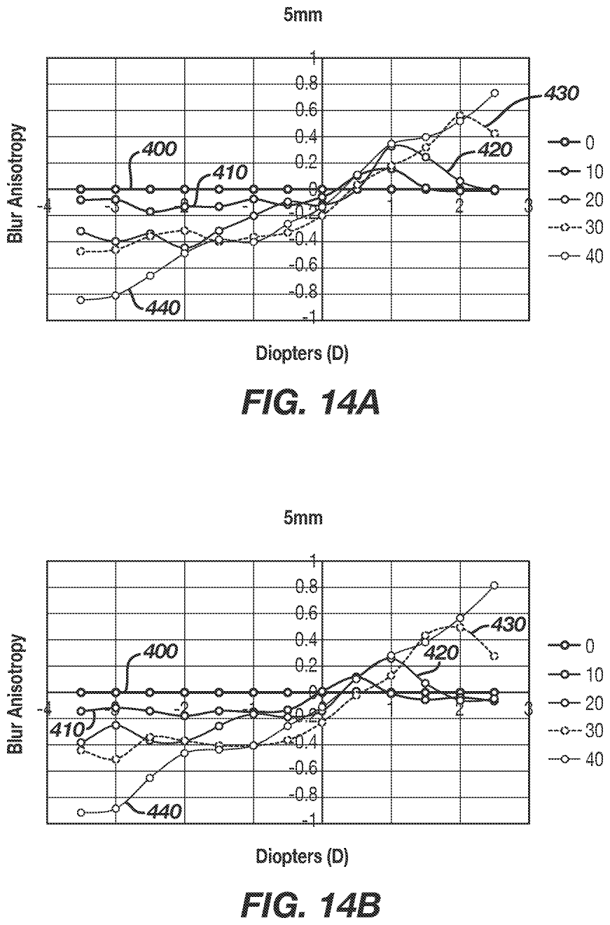

As shown in FIGS. 4a-4d, at peripheral field of views, blur anisotropy shows both a maximum and a minimum value both in front of the retina and behind the retina, which corresponds to the focusing line of astigmatism. For the single vision lens shown in FIG. 4a, the focusing line's magnitude and position at the retina change significantly, and are −0.13, −0.35, −0.41 and −0.89 for 10, 20, 30 and 40 FOV respectively. The FOV averaged blur orientation value at the retinal plane is −0.44 (calculated excluding foveal vision at 0 degrees FOV). The FOV averaged blur anisotropy values of the myopia control lenses of FIGS. 4b, 4c and 4d have higher values of −0.16, −0.18 and −0.11 respectively. FIGS. 14a and 14b illustrate the blur anisotropy values for the lenses of FIGS. 4b and 4d under 5 mm pupil size conditions.

These increasingly more positive FOV averaged blur anisotropy values correlate with increased myopia control efficacy as demonstrated in a clinical study with these same lenses. The clinical study results are summarized below where the single vision lens (SV) corresponds to Lens 1, the “concentric-ring dual focus” lens (DF) corresponds to Lens 3, test lens EE (enhanced efficacy) corresponds to Lens 4, and test lens EV (enhanced vision) corresponds to Lens 2. This clinical study was a multi-national, prospective, randomized, controlled, double-masked, stratified, myopia control clinical trial (NCT03408444). The objective of the study was to compare efficacy and vision of the four lenses. In total, 185 patients completed the study. All study participants were ages 7-12, with each having spherical error within the range of −0.75D to −4.50D, astigmatism <=1.00D, and anisometropia <1.5D. There was no statistically significant difference in baseline characteristics among study participants, as shown in the table below.

| | EV | EE | DF | SV |
|---|---|---|---|---|
| Age [Mean (SD)] | 10.1 | 10.0 | 10.2 | 9.9 |
| | (1.4) | (1.6) | (1.5) | (1.6) |
| % <10 years | 34% | 36% | 38% | 39% |
| Gender (% Female) | 56% | 48% | 60% | 49% |
| Race (% Asian) | 52% | 54% | 52% | 47% |
| Axial Length, mm | 24.42 | 24.65 | 24.31 | 24.43 |
| [Mean (SD)] | (0.86) | (0.78) | (0.67) | (0.79) |
| SECAR [Mean (SD)] | −2.36 | −2.50 | −2.34 | −2.43 |
| | (0.99) | (0.95) | (0.97) | (1.00) |
| % <2.00 D | 45% | 36% | 47% | 45% |

Efficacy of the lenses was determined by both measuring axial length (using a LENSTAR System) and assessing cycloplegic spherical equivalent autorefraction (SECAR) using a Grand Seiko WAM-5500 device, where five repeated measurements were performed, each being the average of 3 consecutive readings. The study concluded that all three study lenses were effective in slowing axial elongation of the eye compared to SV. Efficacy results from this study are shown below.

In FIG. 2A above, the top line represents the single vision (SV) lens, the next line represents the dual focus (DF) lens, the next EV, and the bottom line lens EE. In FIG. 2B above, the top line is lens EE, the next lens DF, the next lens EV, and the bottom line lens SV.

These results indicate that higher blur anisotropy values correlate to better myopia control efficacy. The lenses described herein are designed to generate positive FOV averaged blur anisotropy values rather than the negative values demonstrated in known myopia control (and spherical) lens designs and as opposed to the near zero blur anisotropy targeted in the lenses described in the Yoon patent publication.

At its most general level, the lenses of the present application are able to achieve improved myopia control efficacy in a single vision lens by introducing only variable toric power into the lens. The term toric here is not the more universally applied meaning which is used in optical devices for the correction of central refractive astigmatism. In one embodiment, the variable toricity is described in spatial domain by increasing amounts away from the center of an optical device by the introduction of aspheric surfaces and may generate changes to the oblique astigmatism normally generated in such devices. In another embodiment, the variable toricity is described in angular/FOV domain by increasing amounts away from center FOV by the introduction of aspheric surfaces and may generate changes to the oblique astigmatism normally generated in such devices. It is the toric power profile itself that introduces the myopic control effects. As such, the lenses of the present invention have achieved myopia control efficacy levels with a less complicated lens design than currently available myopia control lenses.

One lens design according to the present invention includes different curvatures along the sagittal and tangential orientations to generate a variable toric power across the lens. For clarity, “variable toric power” as used herein with respect to an ophthalmic lens, denotes a lens having a toric power that varies radially from lens center outward across the optic zone of the lens or at least a portion of the optic zone according to a predetermined toric power profile. The variable toric power may be applied to the front or back surface of the lens as a continuously variable toric power, or may be applied subject to discontinuities at certain radial locations as will be described further below.

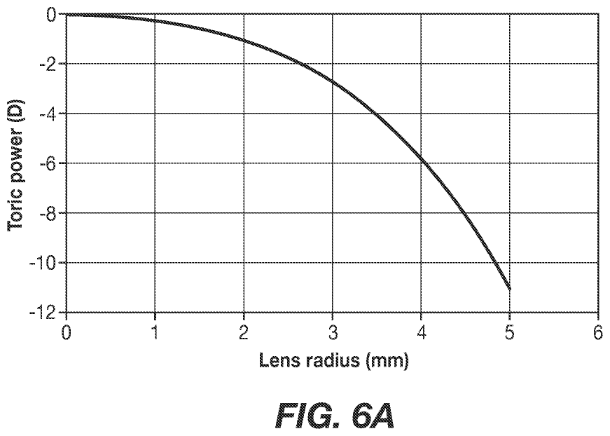

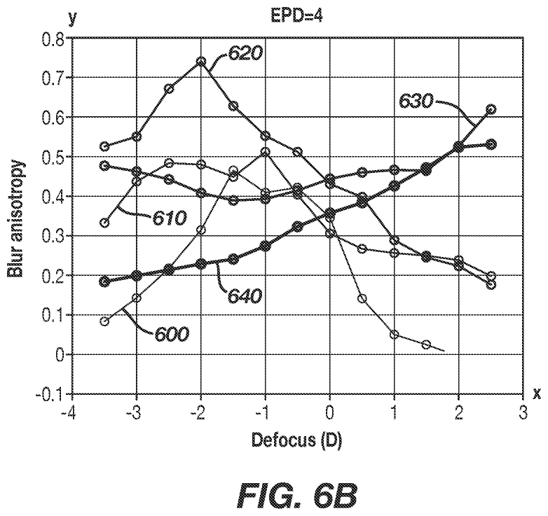

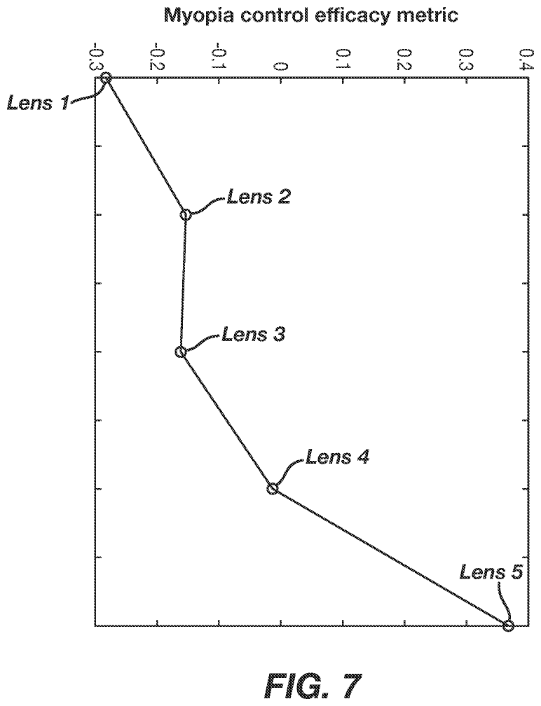

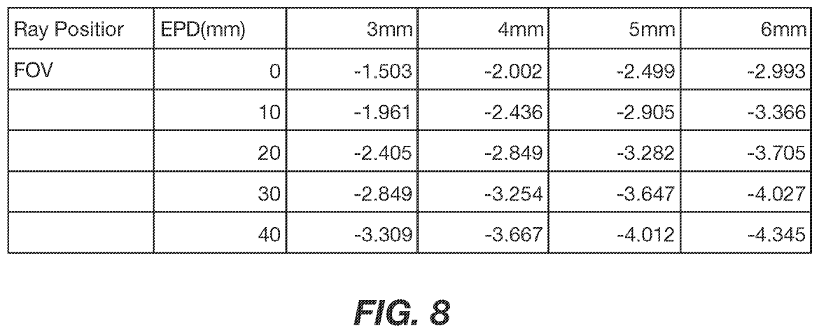

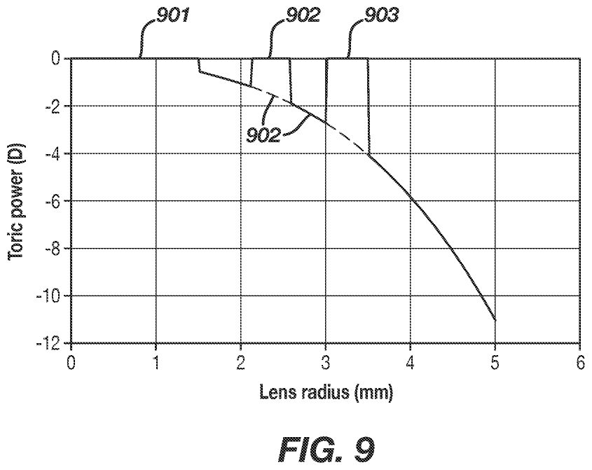

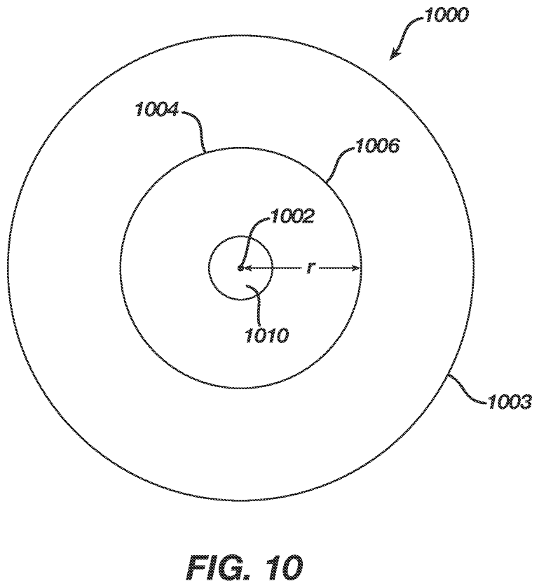

The principles described herein can readily be applied to various types of ophthalmic lenses. In the case of a contact lens, an exemplary lens is shown in FIG. 10. The contact lens 1000 includes a lens center 1002 and a shape defined by a lens outer peripheral edge 1003, and an optic zone 1004 surrounding the lens center and having an optic zone outer periphery 1006. The optic zone of a lens is typically considered the portion of the lens through which a wearer will look during the normal course of wearing the lens to receive intended visual corrections. For example, for a myopic patient the optic zone may have a −3D power to correct the myopic vision of the patient. The optic zone may have a circular configuration defined by a radius (r) from lens center. In the alternative, the optic zone may take any other suitable shape, as may be particularly applicable for spectacle lenses rather than contact lenses. The optic zone may be a single vision optic zone (have a single optical power), or it may include other regions within the optical zone such as a specific myopia treatment region as described further below. Although the diameter may vary, the optic zone in contact lenses is typically designed to be as large as possible without jeopardizing mechanical properties including handling and comfort, and typically falls within the range of 6-10 mm in diameter.

In the case of myopia control lenses, the optic zone may further optionally include one or more additional myopia treatment regions 1010, such as a high ADD power zone at the very center of the lens. Although not necessary for myopia control as described further below, such a region may increase efficacy of the lens as the relative ADD power induces myopic blur such that the focal point falls in front of the retina, and as those skilled in the art readily understand provides a stimulus to move the retina toward the myopic defocus point. This, in turn, provides a counter signal against further lengthening of the eye, or myopia progression.

Although the embodiment above includes a high ADD power region at lens center, any suitable configuration or location of one or more additional myopia treatment regions can be used, whether centrally located or not, and whether on axis (myopic defocus point falls on the optical axis but in front of the retina), or off-axis (myopic defocus is in front of the retina, but does not coincide with the optical axis).

In one embodiment, the contact lens has an aspherical back curvature with a 7.85 radius and a −0.26 conic constant. The front surface has a bi-conic structure specifically designed such that the toric power increases steadily from zero at lens center radially outward to the lens edge as shown in FIG. 5. Significantly, for this exemplary embodiment, the toric power of the lens increases in a predetermined fashion such that the blur anisotropy at or in front of the retina becomes positive. The variable toric power profile illustrated in FIG. 5 is defined by the equation Toric Power=0.0642(r)³−0.1063(r)²−0.018(r), where r is equal to the radius from lens center.

When the blur anisotropy values at 0, 10, 20, 30 and 40 degrees FOV of the above-described lens is modeled in the same way as set forth above for the lenses of FIGS. 4a-4d, the result is shown in FIG. 6 (lines 600, 610, 620, 630 and 640 respectively). As shown, the blur anisotropy values at the retinal plane (x=0) are all positive, as compared to the lenses shown in FIGS. 4a-4d, demonstrating that the lens has induced an opposite blur orientation relative to the comparative lenses.

The efficacy of a lens according to the present invention with induced positive blur as compared to the lenses discussed in conjunction with FIGS. 4a-4d was mathematically tested using the peripheral blur model described above. “Lens 5” is the inventive lens that is identical to Lens 1 (single vision (SV) lens with −3D power) discussed above but having a variable toric power profile described above applied to the lens. The metric used to define myopia treatment efficacy was the FOV averaged blur anisotropy value at zero defocus across degrees FOV: 10, 20, 30 and 40. These average values are shown below:

| | |
|---|---|
| Lens 5 (Variable Toric Lens) | 0.38 |
| Lens 1 (single vision) | −0.44 |
| Lens 2 (EV lens) | −0.16 |
| Lens 3 (dual focus) | −0.18 |
| Lens 4 (Acuvue Abiliti) | −0.11 |

These results are plotted in FIG. 7. As is readily apparent from this figure, the single vision, spherical lens (Lens 1) had the lowest myopia control efficacy as to be expected. Lens 2, 3 and 4 each also had lower myopia control efficacy values than the inventive lens (Lens 5) described herein.

It is noteworthy that the lens with the highest myopia control efficacy is still a single vision lens (Lens 5, variable toric lens as described above), where the only difference between the two single vision Lens 5 and Lens 1 (worst efficacy) is that Lens 5 is a single vision, toric lens having a variable toric power profile, whereas the single vision lens of Lens 1 is a spherical lens. As such, the inventive lens has the surprising result of achieving increased myopia control efficacy using a single vision, but toric lens. Additional “myopia control” regions are not necessary in this embodiment, simplifying design and manufacture of the lenses.

It is to be noted that increasing toric power across a lens may affect image quality, particularly when present in the central vision area. Negative effects on visual quality can be further balanced against increased efficacy if desired. FIG. 8 illustrates for various pupil sizes (3, 4, 5 and 6 mm), the location relative to the center of the lens through which rays enter at various FOVs (0, 10, 20, 30, 40). At 0 degrees FOV paraxial rays enter, for example, a 4 mm pupil across the entire 4 mm. At 10 degrees FOV, however, rays enter the 4 mm pupil at up to 2.436 mm radial distance from lens center. Adverse effects on visual quality may be balanced with myopia control efficacy by leveraging the variable toric power to induce positive peripheral blur outside of the central vision area for a given pupil size. For a 4 mm pupil size, the variable toric power can be applied outside of the 4 mm central optic zone so as to capture only peripheral rays entirely outside of the central vision area.

FIG. 9 illustrates the toric power profile of an exemplary lens where the fundamental principles of the present invention (using variable toric power to induce positive blur) are balanced against degradation in visual quality. In this embodiment, where the average pupil size is assumed to be approximately 3 mm in diameter, no toric power is applied to the lens in this area as illustrated by line 901. For the portion of the lens outside of the 3 mm central optic zone, the toric power can increase steadily from 3 mm to lens edge as shown by line 902. In one embodiment, the variable toric power of the lens is further interrupted at one or more radial zones or regions where the toric power in the lens is again reduced to zero as shown in sections 902 and/or 903. It may be such that for individuals with larger pupil sizes, one or more such zones are further desired to minimize degradation of vision in areas that may still be within pupil zone.

Although the ophthalmic lens described in detail herein is a contact lens, the described and inventive principles can be applied to any ophthalmic lens used for myopia control. For example, the optics described may be applied to a spectacle lens. Further, these principles can also be applied to implantable lenses, such as intraocular lenses or phakic lenses.

### Peripheral Optics Designs

As noted, the results described above indicate that higher blur anisotropy values correlate to better myopia control efficacy. The lenses described above introduce variable toric power to increase blur anisotropy, but blur anisotropy can also be introduced through peripheral optical elements or features as described further below. These designs can be tailored further to provide the desired balance of efficacy and visual acuity.

It is well known that the central vision area of an ophthalmic lens, which is the central viewing area of the lens inside the pupil diameter, is most important to visual acuity. Optical elements or aberrations that disrupt central vision will have a greater impact on visual acuity than optical elements or aberrations that are outside of the central vision area. Although pupil size can vary among the population as a whole, for relatively young children and teens that would benefit most from myopia control lenses, pupil size will typically vary from 2.5-6 mm in diameter under different lighting conditions, with 4.3 mm often being considered the population average for the age subgroup and under a typical room lighting condition.

Currently available myopia control contact lenses introduce myopic defocus areas or zones into the central viewing area of the lens. Although such myopic defocus areas in the central optical region can provide better efficacy, they also degrade vision. For myopia control lenses, designs must balance this degradation in visual acuity against efficacy when defocus is introduced into the central vision area. The present embodiments leverage the principles described above relating to blur anisotropy, to design myopia control lenses having a better balance of efficacy and visual acuity, by introducing myopic defocus optics in a selected manner into the peripheral region of the optic zone.

Referring back to FIG. 10 as an exemplary lens 1000 to which design features described herein can be applied, the lens 1000 includes a lens center 1002 and has a shape defined by a lens outer peripheral edge 1003, and an optic zone 1004 surrounding the lens center and having an optic zone outer periphery 1006. As previously noted, the optic zone of a lens is typically considered the portion of the lens through which a wearer will look during the normal course of wearing the lens to receive intended visual corrections. The optic zone may have a circular configuration defined by a radius (r) from lens center. In the alternative, the optic zone may take any other suitable shape, as may be particularly applicable for spectacle lenses rather than contact lenses. Although the diameter may vary, the optic zone in contact lenses is typically designed to be as large as possible without jeopardizing mechanical properties including handling and comfort, and typically falls within the range of 6-10 mm in diameter. For purposes of describing the present embodiments, the optic zone is further divided into a central optic zone 1010 that surrounds the lens center, and a peripheral optic zone 1020 that surrounds the central optic zone. The central optic zone 1010 is designed to have a diameter that substantially matches the pupil diameter of a wearer, or a typical average pupil size for a population, which typically ranges from 2.5-6 mm with an average in the population of about 4.3 mm.

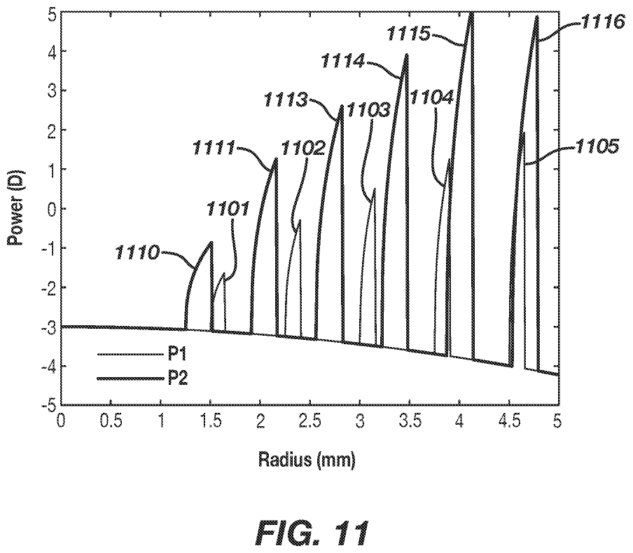

FIG. 11 illustrates the power profiles of two lens designs P1, P2 that balance myopia control treatment efficacy and visual acuity as compared to known lenses that incorporate only central optical elements for myopia control effects. These designs were assessed for efficacy and visual acuity assuming pupil diameters of both 4 mm and 5 mm. As is apparent from FIG. 11, each design includes successive circumferential rings having an optical design that introduces myopic defocus to the wearer of varying ADD powers and at varying radial locations as indicated in the tables below:

Lens P1

| Zone | 1 | 2 | 3 | 4 | 5 |
|---|---|---|---|---|---|
| Radial Distance (mm) | 1.5-1.64 | 2.25-2.4 | 3.0-3.15 | 3.75-3.9 | 4.5-4.65 |
| ADD Power (D) | 1.5 | 3.01 | 4.02 | 4.99 | 6.01 |

Lens P2

| Zone | 1 | 2 | 3 | 4 | 5 | 6 |
|---|---|---|---|---|---|---|
| Radial Distance | 1.2- | 1.91- | 2.56- | 3.22- | 3.87- | 4.53- |
| (mm) | 1.51 | 2.16 | 2.82 | 3.47 | 4.13 | 4.78 |
| ADD Power (D) | 3.0 | 5.92 | 8.01 | 9.84 | 12.0 | 12.0 |

The phrase ADD power, as used herein, reflects the degree (in Diopters) of myopic defocus introduced relative to the base power of the lens. For example, FIG. 11 reflects a base power of −3D correction, and the first myopic defocus zone of lens P1 has an ADD power of about 1.5D relative to the −3D baseline correction. For embodiments P1 and P2, each concentric ring having ADD power to induce myopic defocus, generates myopic defocus as a ring focus around the optical axis rather than as a point focus on the optical axis. Optical designs that generate myopic defocus as a ring around the optical axis, or non-coaxial myopic ring defocus, are described in detail in U.S. Pat. No. 10,901,237, which is incorporated herein by reference in its entirety.

Referring first to the lens design under a 4 mm pupil diameter condition, lens design P1 has only a single circumferential region 1101 within the central optic zone with ADD power that introduces myopic defocus for the wearer, with that ADD power section being a relatively low power of 1.5D. Outside of the central optic zone (within the peripheral optic zone) are successive circumferential zones of increasing ADD power with increasing radial distance from lens center. In the illustrated embodiment, there are 4 additional successive rings (second 1102, third 1103, fourth 1104, fifth 1105 each with the following ADD powers respectively: 3.01, 4.02, 4.99 and 6.01. Under a 5 mm pupil diameter condition, the second 1102 ring will also be positioned within the lens central optic region and only the third, fourth and fifth successive rings will be located in the peripheral optic region or zone.

Referring to the second lens design P2, under a 4 or 5 mm pupil diameter condition, this lens design has at least a first 1110 myopic defocus ring positioned within the central optic zone, and at least a first 1113 and second 1114 myopic defocus rings positioned in the peripheral optic zone. This embodiment may further include a second myopic defocus ring 1111 at least partially positioned within the central optic zone, and may further include third 1114 and fourth 1115 myopic defocus rings positioned in the peripheral optic zone.

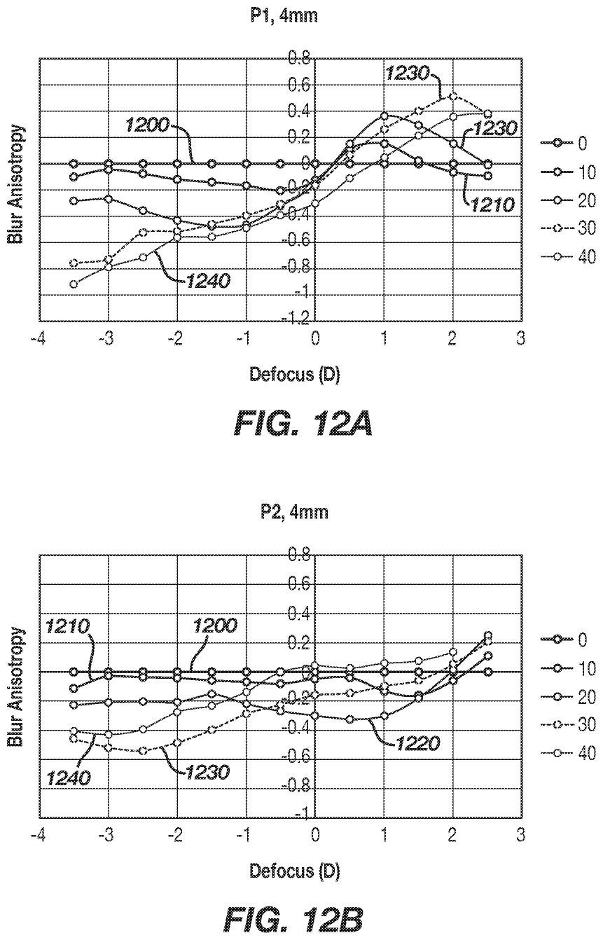

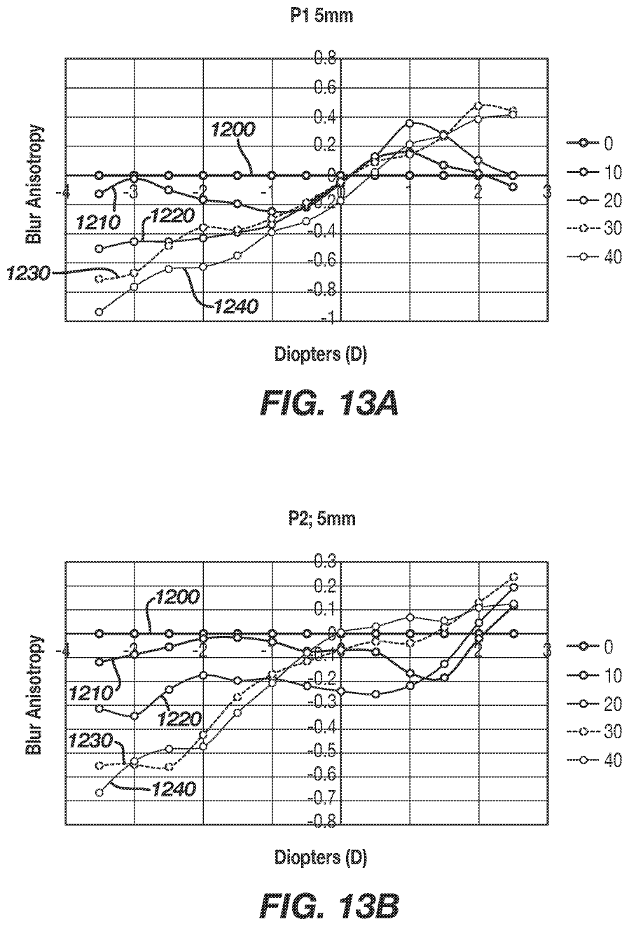

The peripheral FOV model described above as applied to the P1 and P2 lens design generates the blur anisotropy values shown in FIGS. 12a and 12b respectively for a 4 mm pupil size condition and FIGS. 13a and 13b respectively for a 5 mm pupil size condition (reference numerals 1200, 1210, 1220, 1230 and 1240 corresponding to 0, 10, 20, 30 and 40 degree FOV respectively in each figure). For the 4 mm pupil size condition, the focusing line's magnitude and position at the retina are reflected in the table below:

| Degree FOV | P1 Value | P2 Value |
|---|---|---|
| 0 | 0 | 0 |
| 10 | −0.0454 | −0.1291 |
| 20 | −0.2949 | −0.1612 |
| 30 | −0.1592 | −0.1661 |
| 40 | −0.0027 | −0.18978 |

The average blur anisotropy value for the P1 lens is −0.1255 and −0.1615 for the P2 lens. The average blur anisotropy values for the known lenses as described above in conjunction with the toric design (for 4 mm pupil size) are copied again below. The lenses with non-coaxial ring focus are Lens 4, and Lens 6, which is the same as Lens 4 but without the small, high ADD power ring at the very center of the lens which converges on a co-axial focal point in front of the retina.

| | |
|---|---|
| Lens 5 (Variable ToricLens) | 0.38 |
| Lens 1 (single vision) | −0.44 |
| Lens 6 | −0.16 |
| Lens 3 (dual focus) | −0.18 |
| Lens 4 (Acuvue Abiliti) | −0.11 |

It can readily be seen that the P1 lens with an average blur anisotropy of −0.1255 has very similar myopia control efficacy as Lens 4 (−0.11), and better efficacy than Lens 6 (−0.16). The P2 lens efficacy is comparable to Lens 6, but slightly worse than Lenses 5, 3 and 4.

For myopia control lenses it is desirable to provide the best possible balance between efficacy and visual acuity. Based on visual acuity modeling assuming a 4 mm diameter pupil condition, projected visual acuity has been established for lenses P1 and P2, as well as the existing lenses described above having the highest myopia control efficacy (Lens 4 and Lens 6) and is set forth in the table below:

| Lens Design | VA (−10logMAR) |
|---|---|
| Lens 4 | −2.4 |
| Lens 6 | −0.9 |
| Lens 5 | 0.04 |
| P1 | 0 |
| P2 | −1.0 |

Lens P1 has improved visual acuity over both Lens 4 and Lens 6, whereas lens P2 demonstrates better visual acuity over Lens 4 and substantially similar visual acuity as Lens 6. For the lenses discussed above, the table below shows the efficacy and visual acuity values for each for ease of reference.

| Lens | Efficacy | VA |
|---|---|---|
| Lens 4 | −0.11 | −2.4 |
| Lens 6 | −0.16 | −0.9 |
| Lens 5 | 0.38 | 0.04 |
| P1 | −0.1255 | 0 |
| P2 | −0.1615 | −1.0 |

In sum, lens P1 has an efficacy level that is close to that of Lens 4, but with substantially improved visual acuity (almost 2% lines improvement). Lens P1 also has improved efficacy over Lens 6 as well as almost a full line improvement in visual acuity. Lens P2 has slightly worse efficacy than Lens 4, but has almost 1% line improvement in visual acuity, and substantially similar efficacy and visual acuity as Lens 6.

For the 5 mm pupil size condition, the blur anisotropy values are shown in FIGS. 13a and 13b. For comparison purposes, the blur anisotropy values for Lenses 6 and 4 described above assuming a 5 mm pupil size condition is shown in FIGS. 14a and 14b respectively. For the 5 mm pupil size condition, the focusing line's magnitude and position at the retina are reflected in the table below for P1, P2 and Lens 4.

| Degree FOV | P1 Values | P2 Values | Lens 4 Values |
|---|---|---|---|
| 0 | 0 | 0 | 0 |
| 10 | −0.0548 | −0.0703 | 0.0068 |
| 20 | −0.0431 | −0.238 | −0.1391 |
| 30 | −0.0494 | −0.0701 | −0.2263 |
| 40 | −0.1775 | 0.0061 | −0.1123 |

From the table above, the average blur anisotropy for lens P1 is −0.0812, lens P2 is −0.0931, and Lens 4 is −0.1177. As such, both P1 and P2 demonstrate a higher efficacy than Lens 4.

Based on visual acuity modeling assuming a 5 mm diameter pupil condition, projected visual acuity has been established for lenses P1 and P2, as well as the existing lens described above having the highest myopia control efficacy (Lens 4 and Lens 6) and is set forth in the table below:

| Lens Design | VA (−10logMAR) |
|---|---|
| Lens 4 | −2.12 |
| Lens 6 | −0.76 |
| Lens 5 | −0.28 |
| P1 | −0.21 |
| P2 | −1.21 |

Lens P1 similarly has improved visual acuity over both Lens 2 and Lens 4, whereas lens P2 has improved visual acuity of Lens 4 and about a half line worse visual acuity than lens P1 For the lenses discussed above, the table below shows the efficacy and visual acuity values for each for ease of reference.

| Lens | Efficacy | VA |
|---|---|---|
| Lens 4 | −0.1177 | −2.12 |
| Lens 6 | −0.1327 | −0.76 |
| Lens 5 | 0.38 | −0.28 |
| P1 | 0.0264 | −0.21 |
| P2 | −0.0649 | −1.21 |

In sum, lens P1 has improved efficacy over both Lens 6 and Lens 4, and substantially improved visual acuity over Lens 4 (almost 2 lines) and substantially similar visual acuity to Lens 6. Lens P2 also has better efficacy over both Lens 6 and Lens 4, with improved visual acuity over Lens 4 (almost 1 line) and about % line worse visual acuity as compared to Lens 6. The embodiments described above refer to the objective of increasing the positive blur anisotropy, which assumes a horizontal peripheral field of view. While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, may be incorporated with any of the features shown in any of the other embodiments described herein, or incorporated by reference herein, and still fall within the scope of the present invention.

What is claimed is:

1. An ophthalmic lens for at least one of slowing or preventing the progression of myopia, comprising:
a lens center, and a shape defined by a lens outer peripheral edge;
an optic zone surrounding said lens center and having an optic zone outer periphery, the optic zone further comprising a central optic zone surrounding the lens center and defined by a central optic zone radius, and a peripheral optic zone surrounding said central optic zone and extending to the optic zone outer periphery; and
a plurality of myopic defocus zones each positioned at a predetermined radial distance from said lens center, and each introducing a predetermined ADD power, where said predetermined ADD power for said plurality of myopic defocus zones increases with increased radial distance from lens center up to a radius of at least 4 mm;
wherein at least a first myopic defocus zone is positioned within the central optic zone, and at least first and second myopic defocus zones are positioned in the peripheral optic zone.

2. The lens according to claim 1, wherein the central optic zone radius is between 4 and 5 mm.

3. The lens according to claim 1, wherein the at least one myopic defocus zone positioned within the central optic zone is located at least at radial distance of greater than 1.2 mm from lens center.

4. The lens according to claim 3, wherein said at least one myopic defocus zone in the central optic zone has an ADD power of at least 1.5D.

5. The lens according to claim 4, wherein the first and second myopic defocus zones positioned in the peripheral optic zone are located at least at radial distances of about 2.25 mm and 3.0 mm respectively from lens center.

6. The lens according to claim 5, wherein the first and second myopic defocus zones in the peripheral optic zone have ADD powers of about 3.0 and 4.0 respectively.

7. The lens according to claim 6, further comprising a fourth and a fifth myopic defocus zones positioned in the peripheral optic zone.

8. The lens according to claim 7, wherein said fourth myopic defocus zone is positioned at a radial distance of at least 3.75 mm from lens center, and said fifth myopic defocus zone is positioned at a radial distance of at least 4.5 mm from lens center.

9. The lens according to claim 8, wherein said fourth myopic defocus zone has an ADD power of at least about 5D.

10. The lens according to claim 9, wherein said fifth myopic defocus zone has an ADD power of at least 6D.

11. The lens according to claim 6, wherein the myopic defocus zones in the central and peripheral optic zones each cause light passing therethrough to generate non-coaxial myopic ring defocus.

12. The lens according to claim 1, wherein the at least one myopic defocus zone positioned within the central optic zone is located at least at radial distance of greater than 1.2 mm from lens center.

13. The lens according to claim 12, wherein said at least one myopic defocus zone in the central optic zone has an ADD power of at least 3D.

14. The lens according to claim 13, wherein the at least first and second myopic defocus zones positioned in the peripheral optic zone are located at radial distances of at least about 2.56 mm and 3.22 mm respectively from lens center.

15. The lens according to claim 14, wherein the first and second myopic defocus zones in the peripheral optic zone have ADD powers of about 8D and 9.8D respectively.

16. The lens according to claim 15, further comprising a fourth and a fifth myopic defocus zones positioned in the peripheral optic zone.

17. The lens according to claim 16, wherein said fourth myopic defocus zone is positioned at a radial distance of at least about 3.87 mm from lens center, and said fifth myopic defocus zone is positioned at a radial distance of at least about 4.53 from lens center.

18. The lens according to claim 17, wherein said fourth and fifth myopic defocus zones have an ADD power of at least 12D.

19. The lens according to claim 18, further comprising a second myopic defocus zone positioned at least partially within the central optic zone.

20. The lens according to claim 19, wherein the second myopic defocus zone positioned at least partially within the central optic zone is positioned at a radial distance of at least about 1.91 mm from lens center and has an ADD power of at least about 5.9D.

21. The lens according to claim 1, wherein the myopic defocus zones in the central and peripheral optic zones each cause light passing therethrough to generate non-coaxial myopic ring defocus.

22. An ophthalmic lens for at least one of slowing or preventing the progression of myopia, comprising:
a lens center, and a shape defined by a lens outer peripheral edge;
an optic zone surrounding said lens center and having an optic zone outer periphery, the optic zone further comprising a central optic zone surrounding the lens center and defined by a central optic zone radius, and a peripheral optic zone surrounding said central optic zone and extending to the optic zone outer periphery; and
a plurality of myopic defocus zones each positioned at a predetermined radial distance from said lens center, and each introducing a predetermined ADD power,
wherein each of the at least first and second myopic defocus zones positioned in the peripheral optic zone have a larger ADD power than the at least first myopic defocus zone positioned within the central optic zone.

23. The lens according to claim 22, wherein the central optic zone radius is between 4 and 5 mm.

24. The lens according to claim 22, wherein the at least one myopic defocus zone positioned within the central optic zone is located at least at radial distance of greater than 1.2 mm from lens center.

25. The lens according to claim 24, wherein said at least one myopic defocus zone in the central optic zone has an ADD power of at least 1.5D.

26. The lens according to claim 25, wherein the first and second myopic defocus zones positioned in the peripheral optic zone are located at least at radial distances of about 2.25 mm and 3.0 mm respectively from lens center.

27. The lens according to claim 25, wherein the first and second myopic defocus zones in the peripheral optic zone have ADD powers of about 3.0D and 4.0D respectively.

28. The lens according to claim 27, further comprising a fourth and a fifth myopic defocus zones positioned in the peripheral optic zone.

29. The lens according to claim 28, wherein said fourth myopic defocus zone is positioned at a radial distance of at least 3.75 mm from lens center, and said fifth myopic defocus zone is positioned at a radial distance of at least 4.5 mm from lens center.

30. The lens according to claim 29, wherein said fourth myopic defocus zone has an ADD power of at least about 5D.

31. The lens according to claim 30, wherein said fifth myopic defocus zone has an ADD power of at least 6D.

32. The lens according to claim 22, wherein the myopic defocus zones in the central and peripheral optic zones each cause light passing therethrough to generate non-coaxial myopic ring defocus.

33. The lens according to claim 22, wherein the at least one myopic defocus zone positioned within the central optic zone is located at least at radial distance of greater than 1.2 mm from lens center.

34. The lens according to claim 33, wherein said at least one myopic defocus zone in the central optic zone has an ADD power of at least 3D.

35. The lens according to claim 33, wherein the at least first and second myopic defocus zones positioned in the peripheral optic zone are located at radial distances of at least about 2.56 mm and 3.22 mm respectively from lens center.

36. The lens according to claim 35, wherein the first and second myopic defocus zones in the peripheral optic zone have ADD powers of about 8D and 9.8D respectively.

37. The lens according to claim 36, further comprising a fourth and a fifth myopic defocus zones positioned in the peripheral optic zone.

38. The lens according to claim 37, wherein said fourth myopic defocus zone is positioned at a radial distance of at least about 3.87 mm from lens center, and said fifth myopic defocus zone is positioned at a radial distance of at least about 4.53 from lens center.

39. The lens according to claim 38, wherein said fourth and fifth myopic defocus zones have an ADD power of at least 12D.

40. The lens according to claim 39, further comprising a second myopic defocus zone positioned at least partially within the central optic zone.

41. The lens according to claim 40, wherein the second myopic defocus zone positioned at least partially within the central optic zone is positioned at a radial distance of at least about 1.91 mm from lens center and has an ADD power of at least about 5.9D.

42. The lens according to claim 22, wherein the myopic defocus zones in the central and peripheral optic zones each cause light passing therethrough to generate non-coaxial myopic ring defocus.
